(19) United States
(12) Patent Application Publication
(10) Pub. No.: US 2022/0198696 A1
(43) Pub. Date: Jun. 23, 2022

(54) SYSTEM FOR DETERMINING BODY MEASUREMENT FROM IMAGES

(71) Applicant: JONPLE GROUP LIMITED, Radcliffe (GB)

(72) Inventors: ANDREW JONES, MANCHESTER (GB); DMITRY LEYKO, MANCHESTER (GB)

(21) Appl. No.: 17/557,562

(22) Filed: Dec. 21, 2021

(30) Foreign Application Priority Data
Dec. 21, 2020 (GB) 2020308.9

(51) Int. Cl. G06T 7/62; G06T 7/11; G06T 5/00; G06T 7/70; H04N 7/04; A61B 5/00

(52) U.S. Cl. CPC G06T 7/62; G06T 7/11; G06T 5/006; G06T 7/70; H04N 7/04; A61B 5/7275; G01S 17/89; A61B 5/0077; A61B 5/4872; G06T 2207/30196; G06T 2207/20081; G06T 2207/30244; A61B 5/7267

(57) ABSTRACT

A method of determining a body measurement of a subject based on images of the subject is disclosed. A set of images of the subject are received, each image depicting the subject in a respective body pose. For each of a plurality of images of the image set, the method identifies for a given part of the subject body an image measurement of the given body part based on the image, the image measurement defining a two-dimensional extent of the body part derived from the image. The image measurements determined for the plurality of images are then input to a prediction model, the prediction model configured to generate a predicted body measurement of the given body part based on the image measurements.

### CROSS-REFERENCE TO RELATED APPLICATIONS

This application claims priority to G.B. Patent Application Serial No. 2020308.9, entitled SYSTEM FOR DETERMINING BODY MEASUREMENT FROM IMAGES, filed Dec. 21, 2020, which is incorporated herein by reference.

### BACKGROUND

The present innovation relates to systems and methods for determining body measurements from images of a subject. Body measurements play an important part in health monitoring. For example, waist and hip measurements are commonly used to derive a waist-to-hip ratio useful in evaluating and monitoring obesity and weight-loss. However, manual measurement can be difficult and time consuming, and it is generally not possible for a subject to obtain such measurements themselves without assistance. Some attempts have been made to obtain body measurements based on images of a subject. For example, with sufficient images taken of an object, it is possible to construct a three-dimensional model of the object, from which measurements can then be derived. However, accurate volume reconstruction typically requires a large number of images spanning a full 360-degree range of views and taken at precise angular increments. Volumetric reconstruction can also be computationally very demanding. As a result, such techniques are difficult to implement without specialist equipment and outside controlled laboratory conditions.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

One or more techniques and systems are described herein for provide alternative techniques for body measurements that address some of the drawbacks of known approaches.

Accordingly, in a first aspect of the innovation, there is provided a method of determining a body measurement of a subject based on images of the subject. The method comprises receiving a set of images of the subject, where each image depicts the subject in a respective body pose for each of a plurality of images of the image set, and identifying for a given part of the subject body an image measurement of the given body part based on the image. In this implementations, the image measurement comprises a distance measurement pertaining to the body part derived from the image. The method further comprising inputting the image measurements determined for the plurality of images to a prediction model. The prediction model is trained on training data so as to generate a predicted body measurement of the given body part based on the image measurements. Finally, the predicted body measurement is output; where the subject of the body measurement may be, for example, human or animal.

To the accomplishment of the foregoing and related ends, the following description and annexed drawings set forth certain illustrative aspects and implementations. These are indicative of but a few of the various ways in which one or more aspects may be employed. Other aspects, advantages and novel features of the disclosure will become apparent from the following detailed description when considered in conjunction with the annexed drawings.

### BRIEF DESCRIPTION OF THE DRAWINGS

FIG. 1 illustrates a process for obtaining body measurements from images in overview.

FIG. 2 illustrates a set of sub-processes used to implement the process

FIGS. 3B-3E illustrate whole body segmentation and two-dimensional measurements that can be obtained for a set of poses.

FIGS. 4A-4B illustrate body part segmentation models and skeleton models obtained for a set of pose images.

FIG. 5 illustrates a system for implementing described techniques.

FIGS. 6A-6B illustrate a process flow for a system implementing described techniques.

FIG. 7 illustrates a server device and user device for use in the above system.

### DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

The term image measurement as used herein preferably refers to a measurement of a distance in the image or in body structure information derived from and corresponding to the image (possibly after correction or transformation as described below). Such a (two-dimensional) distance measurement may indicate the linear extent (or partial extent) of some feature of the image, such as a linear extent (or part thereof) of an image segment corresponding to a particular body part. As a particular example, the image measurement may indicate a width or half-width of an image segment corresponding to a body part.

The image measurements may be measurements in a measurement plane corresponding to or related to the image plane. Typically, corrections are applied to data obtained from the image to correct for imaging characteristics, and the corrected data is used to obtain the image measurements, so that the image measurements no longer precisely correspond to the coordinate space of the original image but rather to a corrected (e.g. transformed, idealized) version thereof. The predicted body measurement output by the predictor preferably indicates a real-world measurement (or estimate thereof), and may therefore be expressed in real-world measurement units (such as meters), corresponding to a measurement that could be obtained manually e.g. using a tape measure. The prediction model is a machine learning model that has previously been trained on sample data, and serves to translate the image measurements into the real-world body measurement.

The method preferably comprises, for each image, deriving two-dimensional structure information relating to the body pose, and determining the image measurement using the structure information. The two-dimensional structure information may define the structure, shape and/or pose of the subject body as shown in the image. In particular, the structure information preferably comprises one or both of: a skeleton model, the skeleton model preferably comprising one or more skeleton points (e.g. predetermined key points on the body) and/or one or more line segments connecting skeleton points, optionally as vertices and edges of a skeleton graph; and a segmentation model, identifying a plurality of segments of the image corresponding to respective body parts of the subject, wherein the segmentation model preferably comprises a mesh defining contours of respective segments. An image segment is preferably an image region that has been identified by a segmentation algorithm as associated with a particular entity, e.g. body part, whole body, background etc. The skeleton model and/or segmentation model may comprise vectors defining graph/mesh vertices and edges.

Preferably, the method comprises applying one or more corrections to the two-dimensional structure information in dependence on characteristics of a camera system used to obtain the image, wherein the image measurement is determined using the corrected structure information. This can allow the effect of distortions introduced by the camera system to be counteracted, such that distances in the skeleton and/or segmentation model become more accurate representations of real-world distances.

The one or more corrections preferably comprise a correction based on a camera model to correct for image distortions caused by the optical characteristics of the camera system (e.g. lens characteristics). The camera system may be part of a user device, with the one or more corrections comprising a correction based on an orientation of the user device when the image was acquired. The method may comprise receiving device orientation information associated with the image, and applying a correction based on the device orientation information, the orientation information optionally including a gravity vector obtained using one or more sensors of the user device. The one or more corrections are preferably applied to both the skeleton model and the segmentation model.

The method preferably comprises, for each image, applying a segmentation algorithm to the image to obtain the segmentation model for the image by identifying a plurality of image segments corresponding to respective body parts, and identifying the image measurement based on the segmentation model. Performing the segmentation may further comprise performing a whole body segmentation to identify a whole body mask for the image, and preferably refining the segmentation model based on the whole body mask, optionally by constraining segments in the segmentation model to an exterior body contour defined by the whole body mask. The method may further comprise applying a hair segmentation algorithm to obtain a hair segmentation mask identifying one or more image segments corresponding to head hair, and combining the hair segmentation mask with the segmentation model and optionally the whole body mask to produce a refined segmentation model.

Preferably, the method comprises identifying a segment corresponding to the given body part in the segmentation model, wherein the image measurement is determined based on the identified segment.

The image measurement is preferably determined based on a two-dimensional extent (e.g. linear extent) of the identified segment, wherein the two-dimensional extent is optionally a width or half-width of the segment measured in relation to a longitudinal axis of the segment. The image measurement is preferably further determined based on the skeleton model, preferably based on a part of the skeleton model corresponding to the given body part, the method optionally comprising identifying a line segment corresponding to or passing through the body part from the skeletal model, and identifying a measurement line as a line perpendicular to the line segment located at a predetermined location along the line segment and bounded by the segment contour, wherein the distance measurement is determined based on the measurement line (e.g. as the length of the measurement line between opposing points on the segment contour or between the intersection point with the skeletal line edge and a given segment contour point).

The method preferably comprises scaling the image measurements based on a reference measurement of the subject body, preferably a height of the subject, and providing the scaled image measurements as inputs to the predictor model. The reference or height measurement may be obtained based on user input, or based on measurement using one or more sensors of the user device, optionally using a LIDAR sensor. The scaling step may convert the measurements from a dimensionless coordinate system (derived from the image plane coordinate system after corrections have been applied) to a real-world measurement scale, and thus the scaled measurements may be expressed in real-world measurement units e.g. meters.

Preferably, the prediction model receives the image measurements for the given body part determined from each of the plurality of images (preferably after scaling based on the reference measurement) as inputs and outputs the predicted body measurement of the body part.

In preferred implementations, the predicted body measurement comprises a circumference of the body part. However, other types of measurements may be obtained in this manner, e.g. a volume measurement of the body part.

The prediction model preferably comprises a machine learning model trained on a set of training samples, the training samples preferably comprising: image measurements derived from images of a plurality of subjects each in a plurality of body poses, and corresponding measured body measurements of the subjects. For example, the training samples may comprise, for a plurality of subjects, image measurements of a given body part (obtained using the same techniques as described for application of the prediction model, from a set of body pose images), and a real (e.g. manually) measured circumference of the body part. Multiple prediction models may be trained in this manner for different body parts.

Preferably, the prediction model comprises one of: a neural network model, optionally a single-layer perceptron model or a multi-layer neural network model; and a linear predictor model, preferably based on a linear combination of terms, each term comprising a respective image measurement relating to the body part (e.g. from a respective pose image). The method may comprise providing a plurality of trained predictor models, each trained to predict a body measurement, optionally a circumference, for a respective type of body part. The method may then further comprise determining image measurements for each of a plurality of body parts based on corresponding segments in a segmentation model using images from the set of images, and obtaining and outputting a predicted body measurement for each body part using a respective one of the trained predictor models associated with that body part.

The method may comprise determining a plurality of predicted measurements of the given body part using one or more predictor models based on a plurality of image measurements obtained from the images, and deriving volume data, optionally a volume measurement, for the body part from the plurality of predicted measurements determined by the predictor(s). The method may comprise determining a plurality of circumferences of the given body part at different locations on the body part, determining an approximated three-dimensional model of the body part from the plurality of circumferences, and deriving the volume data using the approximated three-dimensional model.

The method may further involve determining derived health data from the body measurement(s) and/or volume data, optionally including a body mass index. The images may be received from an application running on a user device and the body measurement(s) may be output to the application. The method may comprise tracking changes in one or more body measurement(s) or derived value(s) over multiple measurement sessions and outputting change information to a user via the application (e.g. as trend data, graphs etc.).

In a further aspect, the innovation provides a system comprising a server system (e.g. in the form of a server or multiple servers) and a mobile user device having a camera system for acquiring a plurality of images of a subject and transmitting the images and optionally device orientation information to the server system, the server system configured to perform a method as set out above and to output one or more body measurements to the mobile user device. The server system may comprise an application server and an image analysis server.

The innovation also provides a computer readable medium comprising software code adapted, when executed by a data processing device, to perform any method as set out herein, and a system having means, preferably in the form of one or more processors with associated memory, for performing any method as set out herein.

Any feature in one aspect of the innovation may be applied to other aspects of the innovation, in any appropriate combination. In particular, method aspects may be applied to apparatus and computer program aspects, and vice versa.

Furthermore, features implemented in hardware may generally be implemented in software, and vice versa. Any reference to software and hardware features herein should be construed accordingly.

### Overview

Implementations of the innovation provide a system and process for determining body measurements from conventional images of a subject. The images may, for example, be obtained using a smartphone camera or other conventional camera device. The approach is based on obtaining a set of two-dimensional distance measurements from the images, and translating those measurements to estimated real-world circumference measurements of body parts using a set of trained machine learning models.

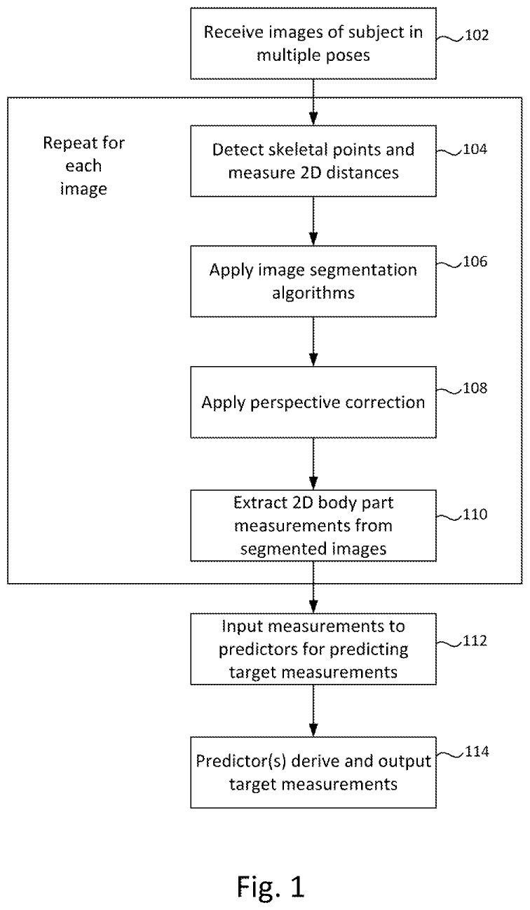

The process is shown in overview in FIG. 1. The process starts in step 102 in which a set of images of the subject are received. The subject is typically a human person, but the described techniques are equally applicable to animals. The image set includes images corresponding to multiple different poses (for example, face-on to the camera with arms lowered or raised, side-on to the camera etc.) A set of processing steps are then performed for each image as follows. In step 104, a skeleton detection algorithm identifies the location of a set of key skeletal points, such as hip, knee, shoulder and ankle joints. Two-dimensional (2D) distances between skeletal points are computed. The two-dimensional distances are distances in the image plane, and may be expressed as pixel distances or in some other unit/scale. In step 106 one or more image segmentation algorithms are applied to segment the body from the image background and to segment individual body parts (such as upper/lower arms, legs, torso, head etc.) Multiple segmentation algorithms may be combined to improve accuracy as described in more detail below.

In step 108, perspective correction is applied to the skeleton data and segmentation data based on the orientation of the camera and characteristics of the camera system. In step 110, a set of basic 2D image measurements (essentially measurements in the image plane but taking into account the perspective corrections) of various body parts are extracted from the segmented images, using the corrected skeleton data and segmentation data. For example, the segmentation may identify a segment of the image—corresponding to a particular region in the image—as an “upper arm” segment. The 2D measurements may include, e.g. height or width of the image segment representing the body part (after perspective correction of the segmentation model).

In step 112, the measurements are provided as input to one or more predictor models. The predictor models determine a set of real-world measurements based on the 2D image measurements derived from the input images. These real-world measurements may be expressed in ordinary real-world measurement units, e.g. meters/centimeters. In preferred implementations, the real-world measurements are circumferences of body parts (e.g. upper arm circumference, waist circumference etc.)

In some implementations, other derived values may be computed based on the outputs of the predictor(s), such as a body-mass-index (BMI). A given predictor model may take 2D image measurements calculated for a set of input images as inputs to generate a single output measurement. The output measurement is thus derived from data obtained from multiple different poses, which can improve accuracy. However, alternatively, predictors may be applied individually for each pose/image, with the output measurements combined (e.g. by averaging or using a further predictor). In preferred implementations, the predictor algorithm(s) comprise machine learning models trained on a set of training samples.

### Image Analysis

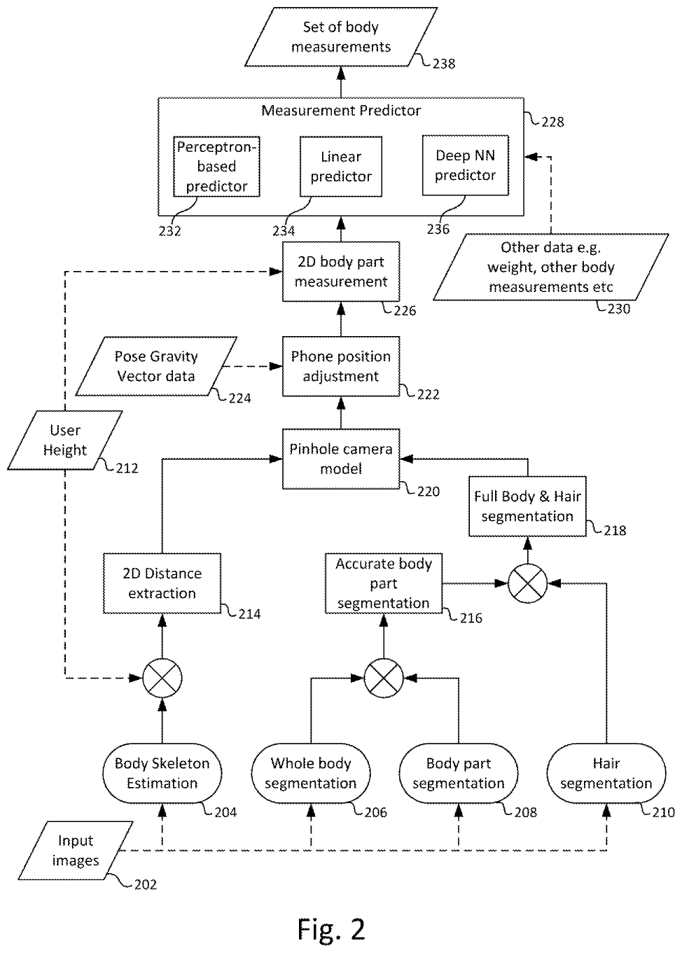

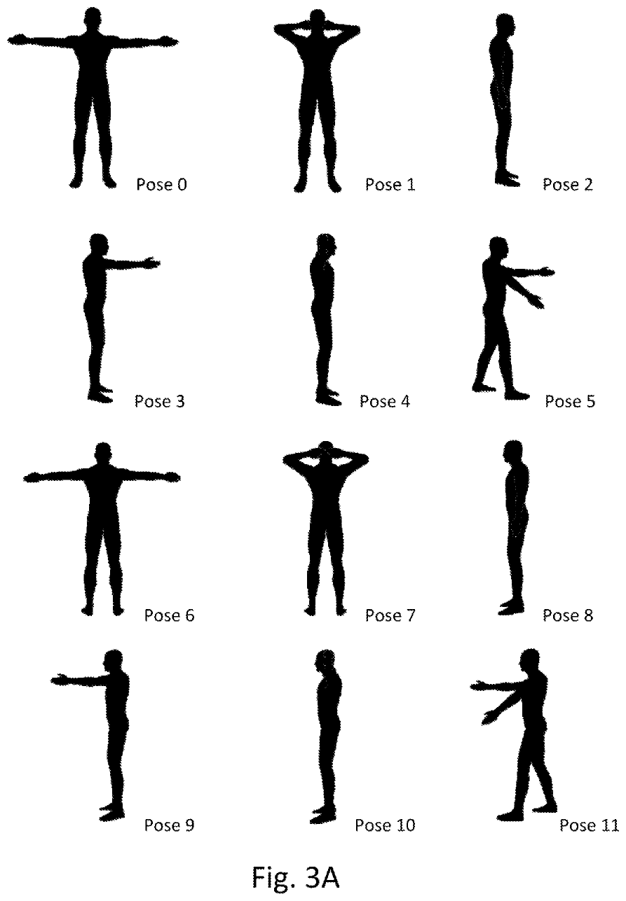

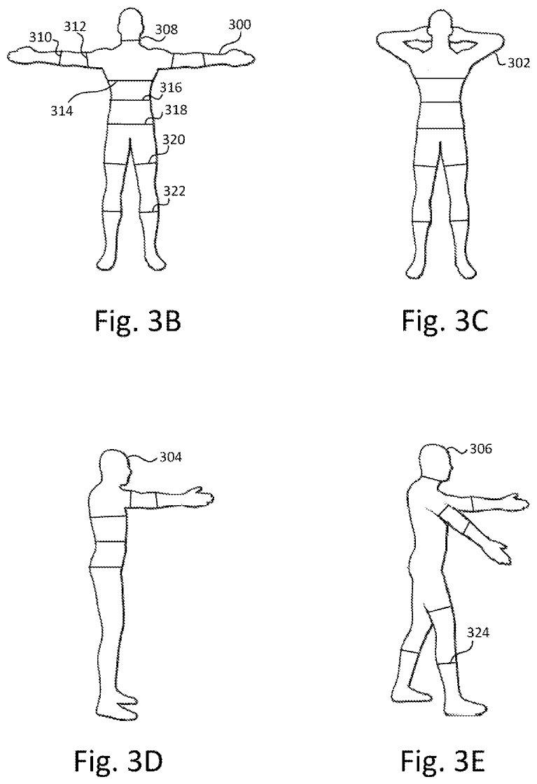

FIG. 2 illustrates the image analysis process in more detail and shows various constituent processing modules used to implement the process, in accordance with an example implementation. The process starts with the set of input images 202, each input image corresponding to a particular pose. Example poses are illustrated in FIG. 3A.

5 A number of pre-processing and segmentation processes are applied to each image as follows.

Firstly, a body skeleton estimation algorithm 204 identifies certain key skeletal points (as described in relation to step 104 above) such as hip, knee, shoulder and ankle joints. The identified points define a simplified “skeleton” as a graph that provides an approximation to the subject's body structure in the particular body pose. The skeletal points (defining vertices of the graph) are processed (operation 214) to identify a set of 2D distances between the skeletal points in step 214. Edges in the graph connect skeletal points and are labelled with the determined distances. Vertices and edges may additionally be labelled with the relevant body part, e.g. joints (such as “elbow”, “shoulder”) for skeletal points and other labels (e.g. “upper arm”) for connecting edges. In an implementation, the skeleton model may be represented as a set of labelled vectors defining skeletal points and connecting line segments.

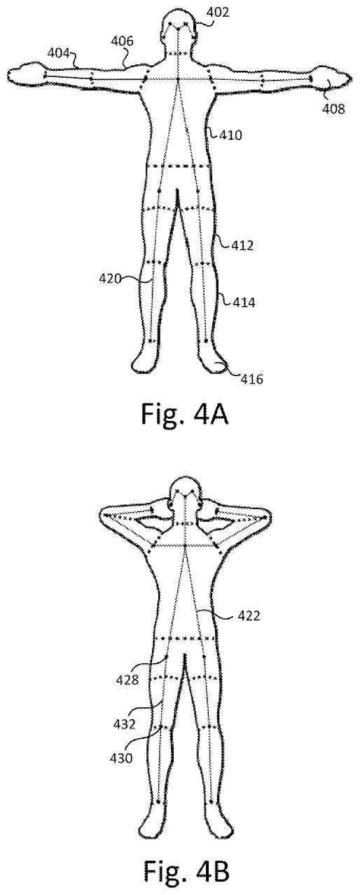

The skeleton estimation may be implemented using known tools, such as the “tf-pose” open-source library. Examples of the identified skeletal graphs for a set of poses are shown in FIGS. 4A-4B. The segmentation processing involves multiple sub-operations, including whole body segmentation 206, body part segmentation 208 and hair segmentation 210. Whole body segmentation 206 segments the image regions representing the subject body from the remaining image regions corresponding to the image background (and any other image content such as other objects, overlays etc.) FIGS. 3B-3E provides a representation of the segmented body shapes for a subset of the poses. This segmentation defines a binary body mask or silhouette, dividing the image into body pixels and background pixels. Segmentation may use a trained machine learning model, e.g. neural network. Existing tools and pre-trained machine learning models may be used to perform the body segmentation, such as the “Deeplab” open-source machine learning-based image segmentation toolset. The output of the segmentation could be a set of vectors defining the body mask contour. Alternatively, a mask image or bitmap could be generated which labels each image pixel as body or background.

In process 208, segmentation of individual body parts is performed. Rather than the binary segmentation into body and background pixels as in process 206, this segmentation identifies individual body regions, corresponding to distinct body parts and outputs a segmented body mask (silhouette). Examples of regions may include head, neck, torso, upper arm, lower arm (forearm), hand, upper leg (thigh), lower leg (calf), and foot. The segmentation labels image regions with the relevant body part label. Labelling may distinguish between left and right versions of a body part, e.g. left hand/right hand etc. Note that the specific listed segment types are by way of example and the specific selection may be varied depending on requirements (e.g. using a single “arm” label rather than separate upper and lower arm labels). Preferred implementations may use a segmentation based on major points of articulation of the human body.

Preferred implementations again use a trained machine learning model to perform the body part segmentation. Some example part segmentations for certain poses are shown in FIGS. 4A-4B. Body part segmentation may be performed using known pre-trained models, such as provided by the “CDCL human part segmentation” toolset. The output of the body part segmentation is preferably a vectorized description of the individual segment contours. However, image masks using different color values to label different segment types could also be used.

The outputs of the whole body segmentation 206 and body part segmentation 208 are combined in merging operation 216 to create an improved body part segmentation. The whole body segmentation 206 can in some cases be more accurate in delineating the body from the background. The merging step overlays the whole body segmentation on top of the body part segmentation to produce a more accurate body part segmentation, by constraining the body part segments to the body contour defined by the whole body segmentation mask.

A further segmentation operation 210 is performed to segment one or more head hair regions of the image, e.g. based on a pre-trained machine learning model. Existing hair segmentation models/toolsets may be used for this step. The process outputs a segmented silhouette of the hair. Using a specially adapted and trained hair segmentation model can improve the accuracy since hair can be challenging to segment correctly for general purpose segmentation algorithms/models. The hair segmentation is then combined with the improved body part segmentation to produce the final body and hair segmentation in operation 218. However, where the body part segmentation is considered sufficiently accurate, the use of a bespoke hair segmentation process could be omitted.

The merging operation 216 can be performed by mask intersection:

body parts mask=body mask ∩ body parts mask

Here, “=” represents the assignment operator. Similarly, the hair segmentation mask can be added to the above result in operation 218 as follows:

body parts mask=body parts mask+hair mask

The full body and hair segmentation 218 provides a vectorized definition of identified body segments, restricted to the whole body contour mask identified by the body segmentation 206. The resulting segmentation model comprises vector descriptions of each individual segment, labelled with the detected body part. The segmentation model is initially expressed in the image plane, e.g. using vectors expressed as pixel distances measured from the image origin (e.g. image location 0,0), or could be expressed in some other appropriate units/coordinate system.

The various pre-processing steps described above result in a skeleton model and a segmentation model for each image, providing two-dimensional structure information describing the body shape and pose.

The skeleton and segmentation models are preferably vector-based descriptions (of the skeleton points and interconnections between them for the skeleton model, and of the body contour and individual segment contours for the segmentation model). The models are in a 2D coordinate space, which may initially correspond to the image coordinate space, i.e. with vectors expressed in pixel distances from an image origin. The models may optionally be scaled to some other interim coordinate space before processing, for example using the user height to scale to real-world measurements. This can be done by determining a scaling ratio between actual user height and height in the image in pixels and using the ratio to scale all model vectors. Alternatively, some other coordinate space could be used, or the models could remain in the image coordinate space. The rescaling may occur after extraction of the body skeleton (204) and prior to 2D distance extraction (214), and similarly after segmentation steps 206, 208, 210 (or alternatively after the complete segmentation model 218 has been obtained). Subsequent processing e.g. in steps 214, 220, 222 is then performed in the interim coordinate space. Note that, regardless of the interim coordinate space used, the final 2D measurements (step 226) are obtained from the corrected model (see below) and are rescaled again as described later.

Operations 220 and 222 together perform corrections to the skeleton model and segmentation model reflecting characteristics of the imaging system. The corrections use a coordinate mapping matrix to map from the camera coordinate system to a real-world coordinate system, correcting e.g. for camera/lens characteristics and device orientation.

In particular, in operation 220, the segmentation model and the skeletal model with identified 2D distances between skeletal points are processed using a camera model to adjust for characteristics of the imaging subsystem and to combine and cross-reference the two data sources. This involves overlaying the models after applying the camera model adjustments. For example, a typical camera will not represent distances evenly across an image (e.g. distances may become increasingly distorted moving out from the center of the image). Application of the camera model applies corrections to the segmentation model and skeletal model to account for these effects. The camera model is preferably based on a pinhole camera model. The required corrections may be obtained in a calibration step for the specific device/camera model.

Operation 222 performs further adjustments to account for the orientation of the user device to ensure that the device's (and hence camera's) orientation during capture does not affect the accuracy of the result. In particular, if the camera device was not held straight during image capture, then the image plane would not be parallel to the human body shape being imaged. The resulting distortion is corrected in this step.

The adjustment uses orientation data 224 from the source device (e.g. smartphone). This may be in the form of a gravity vector obtained by the smartphone using orientation sensors (e.g. using one or more accelerometers), which specifies the orientation of the phone relative to the Earth surface. A separate gravity vector is recorded for each pose image by the smartphone and associated with the pose image (e.g. as image metadata). The correction adjusts the vectors of the skeleton and segmentation models to eliminate the inaccuracies introduced by the device orientation.

The operations 222 and 224 essentially transforms the structural information (skeleton and segmentation models) from the image plane (e.g. as a set of pixel distance vectors) to an idealized version of the image plane and as such the information is no longer expressed in the coordinate system of the original images after correction but can be considered to be expressed in a new, dimensionless coordinate system (possibly rescaled e.g. based on user height as discussed earlier).

In operation 226, 2D image measurements of individual body parts are obtained from the corrected skeleton model and segmentation model. Each individual body part may be associated with one or more 2D image measurements. For example, a width of each body part may be measured. Examples of measurements are indicated in FIGS. 3B-3E and discussed in more detail below. The 2D image measurements are based on the segment model (comprising the segments corresponding to body parts) and skeleton model (e.g. the key skeletal points and distances between them). They are referred to herein as “image measurements” or “image-derived measurements” since they are obtained directly from structural information (segmentation and skeleton) extracted from the image, though the perspective corrections mean that the image measurements do not necessarily correspond to (scaled) pixel distances in the image plane of the source image.

In one approach, for a given body part corresponding to a particular segment in the segmentation model, a location at which a width of the body part is measured is determined from a corresponding line segment in the skeleton graph, connecting two skeletal points and defining a longitudinal axis of the body part. The measurement location can be selected e.g. as the center of the line segment, or based on some other location along the line segment (e.g. specified as a relative/proportional location relative to the length of the line segment). A perpendicular measurement line segment is then computed which intersects the skeletal line segment at the selected location and is bounded by the segment contour (as given by the segmentation model). The width measurement is then determined based on the measurement line, e.g. as the length or half-length of the measurement line (e.g. measured between the two opposing points on the contour or between the selected location on the line segment and a point on the contour to one side of the line segment).

As an example, the algorithm may identify the upper arm in the skeleton graph as the line segment (edge) connecting the vertices corresponding to the shoulder and elbow joints. The width of the upper arm segment identified through segmentation can then be measured as the extent of that segment along a transverse (perpendicular) line intersecting the upper arm line segment halfway along its length (or at some other predetermined point in relation to the edge or vertices). The resulting 2D image measurements are in the dimensionless coordinate system of the idealized image plane (corresponding to the image plane after application of the corrections), or a scaled version if scaling has been performed e.g. based on user height.

In a preferred implementation, these measurements are then scaled based on the height of the subject to express the information as real-world measurements, e.g. in units of meters/centimeters. For example, a scaling factor may be determined as a ratio between the subject's real height, and the height as given by the corrected skeleton/segmentation models, and used to scale the obtained measurements. The height may have been supplied as input by the user, or may be detected through some other means, as discussed further below. Note that this scaling is independent of the initial scaling described above (if performed). The initial scaling simply provides an interim coordinate space in which to work. This second scaling operation uses the corrected models (after application of the camera model and orientation correction) and thus can provide more accurate results. Note also that the second scaling operation is unaffected by whether the initial scaling operation was height-based or based on some other reference coordinate system, or was omitted.

The (scaled) 2D image measurements obtained are provided as input to a set of predictors, preferably in the form of trained machine learning models. A separate predictor is provided for each body part of interest. Each predictor takes as input the set of 2D image measurements obtained from all the different poses for a given body part and outputs a corresponding desired real-world measurement. The target measurement is preferably the circumference of the body part. The target measurement is preferably expressed in a real-world measurement scale/unit, e.g. meters/centimeters.

Each of the predictors has multiple inputs which correspond to the 2D image measurements for the particular body part obtained for different pose images. Different body part predictors may have different sets of inputs, since not every pose may be suitable for every body part measurement. Thus, a predetermined set of 2D image measurements for a relevant set of pose images forms the input for a given body measurement predictor. Each predictor is preferably trained offline using training samples, where the training samples correspond to 2D image measurements derived from pose images using the FIG. 2 process, together with corresponding actual real-world body part measurements (e.g. circumferences), which may have been obtained manually (e.g. using a tape measure).

The predictors thus output one or more real-world measurements for each body part of interest, e.g. a circumference for each body part. Note that measurements need not necessarily be determined for every body part identified in the segmentation. For example, where the system is used to obtain health characteristics of a user, only particular body measurements may be of interest, e.g. chest, waist, leg and arm circumferences, whereas other body parts (e.g. hands, feet) may be considered less important and thus no analysis may be performed for those body parts. The resulting set of body measurements 238 can be provided as output to a user or be further processed, e.g. to derive other information such as a body-mass index, waist-to-hip ratio etc.

A variety of machine learning techniques may be used to create the predictor models 228. Examples include:
Perceptron based predictors 232: These use a small (typically single-layer) neural network that extrapolates the results based on weights and biases that were adjusted during the training process. This approach is computationally simply but may in some cases have a tendency to overfit the data.
Linear predictor models 234: These can be based on an ellipse model, or using a simple linear combination of terms corresponding to image measurements, by using weights and biases that are adjusted during the training process.
Deep neural network models 236: This approach involves constructing a deep neural network. Such a neural network may use additional inputs (e.g. subject data such as weight, other known body measurements, height etc.), and may be able to cross-correlate more data to potentially produce a more accurate and reliable result than the above described techniques, though at the cost of greater computational complexity and a substantially larger training data set.

Other machine learning models could be employed, e.g. decision trees, random forest models etc. In one approach, a linear predictor may be implemented using the following linear prediction model:

l=C₁d_{frontal}+C₂d_{side}

Where l is the required circumference estimate, d_{frontal} is some frontal estimate of body measurement and d_{side} is some side estimation of the body measurement. For example, with reference to FIGS. 3B-3E, d_{frontal} could be the 2D calf measurement 322 in the FIG. 3B pose, and d_{side} could be the corresponding measurement 324 in the side view pose of FIG. 3E. The predictor coefficients C₁ and C₂ are obtained during training of the predictor. While the above example is based on just two 2D image measurements, in practice for a given body part there may be more poses that allow 2D image measurements to be obtained for the body part. In that case, additional terms can be added to the predictor, so that the linear predictor includes a separate term (with respective coefficient and 2D image measurement) for each pose image in which the body part is measured.

As mentioned above, instead of a linear predictor a neural network may be employed. In that case, the different measurements of a particular body part (e.g. d_{frontal} and d_{side}, though in practice there may be more measurements from a larger range of body poses) are provided as inputs to the neural network, which outputs the estimated circumference of the body part, based on a set of weights obtained during the training of the neural network.

FIG. 3A illustrates a set of poses that may be used as the basis for the measurement algorithm. FIGS. 3B to 3E illustrate whole-body segmented images obtained for a set of four different poses, each showing a respective subject silhouette 300, 302, 304, 306. These are obtained by the whole-body segmentation 206. FIGS. 3B to 3E also illustrate 2D image measurements derived by the body part measurement step 226, as a set of measurement lines spanning across various body parts. For example, FIG. 3B illustrates a neck measurement 308, forearm measurement 310, upper arm measurement 312, chest measurement 314, waist measurement 316, pelvis measurement 318, thigh measurement 320 and calf measurement 322. The measurements obtained may be the whole length or partial length (e.g. half length) of the illustrated measurement lines. As shown in FIGS. 3B to 3E, different poses may provide different sets of 2D body measurements (e.g. arm measurements are not obtained from the FIG. 3C pose).

The illustrated poses and measurements are purely by way of example and may be adapted to the requirements of particular implementations. Some concrete examples of how measurements may be derived as various straight line distances are discussed further below.

FIGS. 4A and 4B illustrate the body part segmentation resulting from operations 206, 208, 210, 216. The segmentation is shown by way of dotted lines. For example, FIG. 4A shows head segment 402, forearm, upper arm and hand segments 404, 406, 408, torso segment 410, and upper leg, lower leg and foot segments 412, 414, 416.

FIGS. 4A and 4B also illustrate the skeleton graphs obtained by the skeleton estimation algorithm 204. Each pose is associated with a distinct 2D skeleton graph 420, 422. The graphs comprise a set of skeletal points (e.g. 428, 430) as vertices of the graph, interconnected by graph edges (e.g. 432). Many of the skeletal points correspond to major joints (i.e. major points of articulation) of the human skeleton, but other skeletal points may also be used, e.g. points corresponding to nose/brow/ears as visible within the head segment. Similar segmentations and skeletal graphs are generated for the other pose images, including side views.

### Body Measurement System and Mobile Application

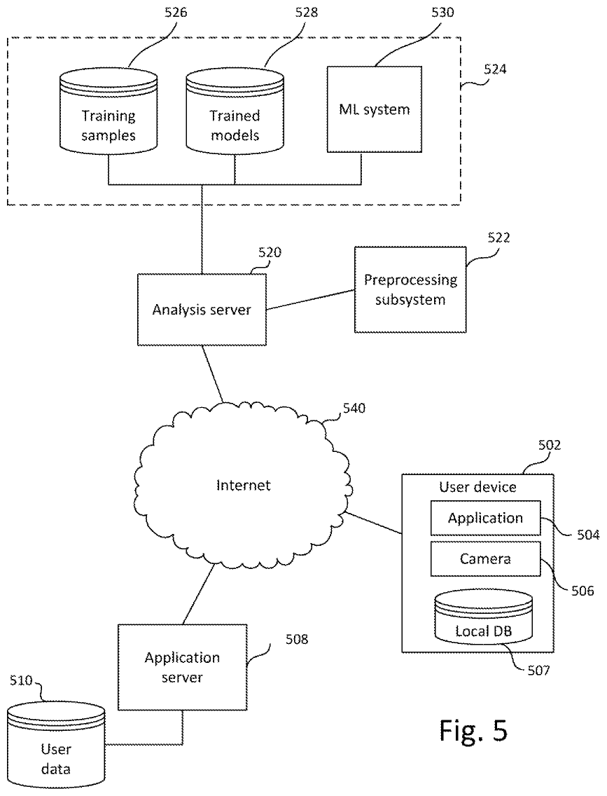

In some implementations, the above body measurement system is integrated into an application and service for providing measurement and health information to users. FIG. 5 illustrates a system for providing such a service to user devices.

The service is implemented by way of a mobile application (app) 504 running on a user device 502, for example a smartphone, tablet computer or other personal computer/communications device. The mobile application 504 implements the client side processing and user interface for the service and communicates over one or more networks, e.g. including the Internet 540 (and mobile telecommunications networks as needed) with an application server 508, which implements any server-side processing, data storage etc. The user device 502 also includes a camera 506 for acquiring images of a subject in various poses, and a local database 507 for storing application data locally, such as images, measurements, user data etc.

Note that instead of a bespoke native application, the service could be implemented as a web service, with the application 504 comprising a web browser communicating with a web server as the application server 508.

The application server 508 is connected to a database 510 of user data, including for example user account data (e.g. user identifiers, passwords, personal information, past measurement data etc.) Note that the user data database 510 could be integrated into the application server or provided as an external database/storage server.

An analysis server 520 is also provided which performs the image analysis as discussed in relation to FIGS. 1 and 2. The analysis sever includes, or is connected to, a pre-processing subsystem 522 and a measurement predictor subsystem 524. The pre-processing subsystem performs the various pre-processing steps and algorithms 204, 206, 208, 210, 214, 216, 218, 220, 222 and 226 shown in FIG. 2 (corresponding to steps 104-110 of FIG. 1) to produce a set of 2D image measurements. The pre-processing system may perform other conventional image processing steps to improve the images prior to analysis, e.g. contrast enhancement, straightening, color correction etc.

The predictor subsystem 524 receives the 2D image measurements and outputs corresponding estimated (predicted) real-world measurements using a machine learning system 530 based on a set of trained models 528 that were trained offline using a set of training samples 526. The predictor subsystem 524 thus implements process 228 of FIG. 2 (corresponding to step 112-114 of FIG. 1).

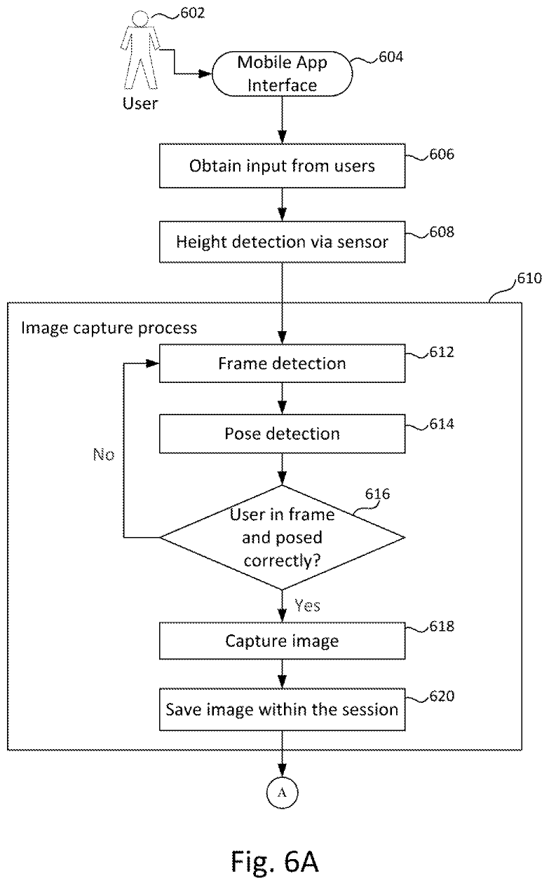

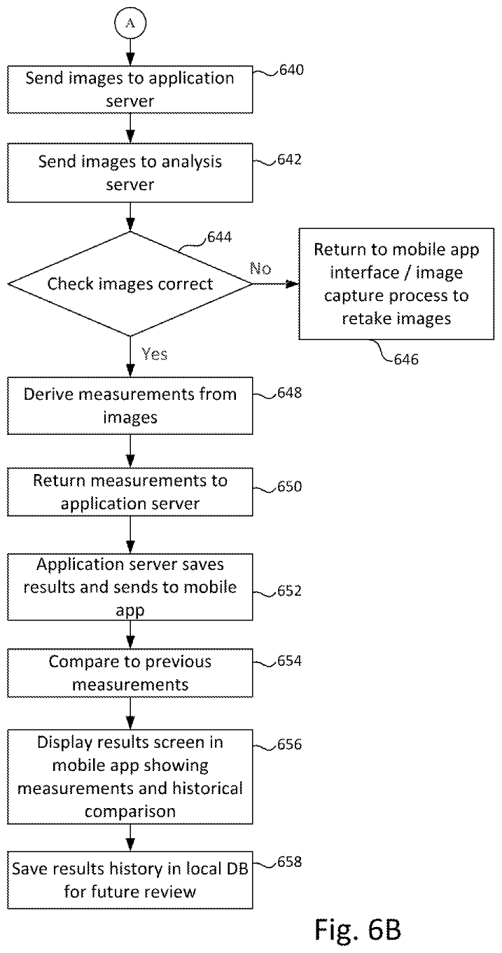

FIGS. 6A-6B illustrate an example of a process flow implemented in the system of FIG. 5 to perform image analysis and measurement derivation based on images acquired by a user device. The process starts with a user 602 interacting with the mobile application interface 604 to start the measurement process. In step 606, the application obtains various inputs from the user, such as name, email address, age, gender etc. This information may be stored in a user profile in local database 507 and/or user data database 510 at the application server so that the user is only asked to input the information the first time they use the application.

In step 608, height detection may be performed. Height detection may be performed using the device camera, e.g. using augmented-reality (AR) techniques. Various approaches may be adopted; in one example, the user may be prompted to take an image in a particular pose (e.g. facing the camera) whilst holding an object of known, standardized dimensions, such as a credit card to allow the scale of the image to be determined and the height of the subject to be calculated. In other examples, height may be detected using LiDAR (laser imaging, detection, and ranging) where the user device includes a LiDAR sensor. A neural network based height detection algorithm may also be employed. Height detection may be performed every session or just on first use, with the height stored in the user profile. Instead of automatic height detection, the user may input their height as part of step 606, or separately.

The process then proceeds to the image capture process 610. This may be repeated a number of times for different poses. The application may use a fixed set of poses, for example the set of poses shown in FIG. 3A, with the application obtaining images for each pose. For a given pose, the required pose may be displayed to the user on the user device screen, e.g. as a graphic. The image capture process detects frames acquired by the camera (step 612) and identifies the user pose. For example, this may use a local version of the segmentation algorithm 206 to identify a silhouette which the system may match against the required pose. If the user is identified as visible in the frame and posed correctly (step 626), then the image is captured (618), and saved as part of the image set for the current session (step 620). However, steps 612 to 616 could be omitted, with the system relying on the user to capture suitable images for the required poses, in which case the system could reject unsuitable images at a later stage of processing.

The orientation of the phone is recorded (e.g. in the form of the gravity vector data 224) at the same time and is stored with the image or as part of the image (e.g. in image metadata). To improve accuracy and simplify processing, the application may require the orientation of the device to be within defined bounds, e.g. so that it does not deviate from vertical orientation by more than a threshold. The application may thus check the device orientation obtained using device sensors, and display a message if the orientation is unsuitable, and require the user to retake the image (or prevent the image being taken in the first place). The application may similarly enforce other requirements, such as suitable lighting conditions.

The process may be repeated until the full set of poses have been successfully acquired. Alternatively, the system may require a minimum set of pose images to be acquired but may give the user the option of acquiring images for additional poses to improve accuracy of the final measurements.

Once the set of pose images has been acquired, the process continues on FIG. 6B, where the images are sent to the application server 508 in step 640 for storage. Transmission of the images may be in response to explicit user request, e.g. by clicking a “submit images” button. At this point, control transfers to the application server. The application server then triggers an API call to the analysis server 520 and transmits the images to the analysis server in step 642.

Control then transfers to the analysis server, where the following steps are performed. In step 644 the server performs checks to determine whether the images are suitable for analysis (e.g. checking that a human figure is visible and that there are sufficient distinct poses and/or the correct poses represented, that the image quality is sufficient etc.). Some or all of these checks could alternatively be carried out at the user device. If the images are not suitable then control passes back to the mobile application to repeat the image capture process in step 646.

If the images are suitable, then in step 648 the image analysis is performed, and the body measurements are derived from the images using the process of FIGS. 1 and 2. The set of measurements (e.g. a set of circumferences for different body parts) are returned to the application server 508 in step 650. The server stores the measurements in the user profile within the user database 510 and transmits the measurements to the mobile application (step 652).

At the mobile application, measurement results are displayed and further analysis, e.g. historical comparison, may also be carried out. In one example, the application may compare the measurements to previous measurements to identify any changes—e.g. increases or decreases in particular body measurements (step 654). The application may also compute derived quantities such as estimated body mass index (BMI) or changes in such derived quantities. The application then displays one or more results screens showing the measurements and possibly any historical comparisons in step 656. For example, trend graphs of measurements or derived quantities over time could be displayed. Additionally, the measurements and other analysis results are saved in the local database 507 in step 658 for future review by the user.

The application may provide the measurement analysis and any historical measurement comparisons, derived metrics etc., as part of fitness, diet, or health improvement program. For example, the application could make lifestyle recommendations, and use the body measurement functionality and historical tracking of body measurements to track the user's progress on a weight loss program.

Note that for security and privacy reasons, neither the application server nor the analysis server permanently stores the pose images. The images are only stored temporarily in memory (e.g. RAM) whilst they are being forwarded by the application server and processed at the analysis server and are deleted after being processed. While generally described in relation to body measurements for human subjects, the described techniques may also be applied to animal subjects, e.g. in agricultural and veterinary contexts (e.g. to track animal growth or health).

### Extensions to Obtain Volumetric Information

In the above examples, the prediction system determines estimated real-world circumferences of body parts from the image-derived 2D measurements. However, the techniques may be adapted to obtain information on body volume or mass, or other body characteristics. In one example, the prediction models could predict volume information directly, e.g. by predicting a volume or mass of a body part from the 2D image measurements instead of circumferences.

In one implementation, the prediction models are used to obtain multiple circumference measurements of a body part. For example, circumference measurements could be obtained at locations along an upper arm segment, e.g. at ⅓, ⅔ and 3/3 along the longitudinal axis of the upper arm segment, based on 2D image measurements obtained at corresponding image locations. The number of circumferences determined can be varied based on requirements, e.g. trading off computational complexity against accuracy.

Interpolation between the predicted circumferences can then be used to obtain a complete approximated 3D model of the body part, from which volume information (e.g. a volume measurement) is then calculated.

### Further Implementation Details

The following sections provide additional detail on how the above techniques may be implemented in an example implementation.

### Segmentation and Pose Estimation

In an implementation, the pose/skeleton estimation (see box 204 in FIG. 2) uses “tf-pose”, a pre-trained neural network based solution, available at:

https://github.com/ildoonet/tf-pose-estimation

This detects the following human body skeleton points: nose, neck, left/right shoulders, left/right elbows, left/right wrists, left/right hips, left/right knees, left/right ankles, left/right eyes and left/right ears.

Segmentation is based on a neural network based approach as described above as these approaches are typically more robust to different conditions of illumination and human body position and forms. The approach uses multiple pre-trained neural network models as illustrated in FIG. 2.

The human whole body segmentation 206 uses Deeplab, available at:

https://github.com/tensorflow/models/tree/master/research/deeplab

This segments the image to produce a human body mask corresponding to the whole body.

Body part segmentation 208 uses CDCL, available at:

https://github.com/kevinlin311tw/CDCL-human-part-segmentation

This segments human part masks: head, torso, left/right upper arms, left/right forearms, left/right hands, left/right thighs, left/right shanks, left/right feet. The segments are labelled with the appropriate body part.

Hair segmentation 210 uses the following pre-trained neural network solution:

https://github.com/ItchyHiker/Hair_Segmentation_Keras

This identifies a human hair mask.

As shown in FIG. 2 the segmentation outputs of the above neural network models are combined. Deeplab provides more accurate body segmentation but does not distinguish body parts so mask intersection is performed to combine the masks:

body parts mask=body mask ∩ body parts mask

The hair segmentation mask is then added to the above result:

body parts mask=body parts mask+hair mask

After that, for each image the pre-processed data contains body parts masks and skeleton points (as illustrated e.g. in FIGS. 4A-4B).

### 2D Body Part Measurement

The body part measurement extraction uses a set of heuristic algorithms (since the neural network segmentation returns only indirect information about distances). The body part measurement obtains 2D distances in the image plane, e.g. as distances in pixels (after perspective correction).

The measurement extraction is based on the body part segmentation and the skeleton/pose detection. In an implementation, the 2D measurements are extracted based on the body part segmentation for the following body parts: right/left calf, right/left thigh, right/left bicep, right/left forearm, neck, chest, waist and hips. In each case the width of the body part is measured.

Not every pose may be suitable for each measurement. In an implementation, the following measurement sets are obtained for each of a set of predefined poses:
Pose0—right/left calf, right/left thigh, right/left bicep, right/left fore-arm, neck, chest, waist, hips
Pose1—right/left calf, right/left thigh, chest, waist, hips
Pose2—neck, chest, hips
Pose3—right bicep, right forearm, chest, waist, hips
Pose4—chest, waist, hips
Pose5—right/left calf, right thigh, right biceps, right/left forearm, neck
Pose6—right/left calf, right/left thigh, right/left bicep, right/left fore-arm, neck, chest, waist, hips
Pose7—right/left calf, right/left thigh, chest, waist, hips
Pose8—neck, chest, hips
Pose9—left bicep, left forearm, chest, waist, hips
Pose10—chest, waist, hips
Pose11—right/left calf, left thigh, left bicep, right/left forearm, neck

The idea underlying each heuristic algorithm is that 2D distances are determined for spans that are perpendicular to human bones or spine and limited by the body part masks contour (as in the examples of FIGS. 3A-3D). The identified skeleton points allow assumptions to be made about the locations of human bones (e.g. corresponding to main longitudinal axes of body parts) and together with the body part masks given by the segmentation are used to obtain the 2D image measurements based on empirically obtained measurement criteria. The following gives some concrete examples.

In the following examples, measurement line segments are determined transverse to a particular body part axis. In each case, the line segment is bounded by the body mask/body part mask obtained through segmentation, and the extent of the bounded line segment (or a part thereof) provides the desired 2D image measurement. The fractional numbers in the examples indicate relative locations along a line segment (relative to the total line length which is defined as 1); thus a location 0.5 is halfway along a line segment and a sub-segment extending over range [0.4:0.6] defines a sub-segment extending from a point at 40% of the line length to a second point at 60% of the line length between the end points.

Thigh Measurement:
On the skeletal line segment formed by the appropriate hip and knee points take a point that divides the line segment in the proportion of 0.6.
For this point, calculate a perpendicular line segment bounded by the body part mask (defined by the segmentation model). The length of that line segment is the desired 2D distance.

Calf Measurement:
On the line segment A formed by the appropriate ankle and the knee points, select the sub-segment B in the range [0.4:0.6].
Split segment B into points with a distance of 1 pixel.
For each point, draw a perpendicular line segment C bounded by the body part mask.
Depending on the view measure the length:
   from the point to the end of segment C for the side views towards the back of the calf, or
   of the segment C (for front/back views).
The segment with maximum length is used as the output measurement.

Neck Measurement:
For the front view the algorithm is as follows:
   On the line segment A formed by the neck and the nose points, extrapolate the segment B in the range from the neck point to the upper point of the torso mask.
   Split segment B into points with a distance of 1 pixel.
   For each point, draw a perpendicular line segment C.
   Measure the length of the segment C. The minimum length segment is selected.
For the back view the algorithm is as follows:
   On the line segment A formed by the neck point and the middle point between the ears, select the line segment B in the range from the neck point to the upper point of the torso mask.
   Split segment B into points with a distance of 1 pixel.
   For each point, draw a perpendicular segment C bounded by the body mask.
   Measure the length of the segment C. The segment with minimum length is the desired one.
For the side views the algorithm is describe as follows:
   On the segment named A formed by the neck point and the appropriate ear point, select the segment named B in the range from the neck point to the upper point of the torso mask.
   Split segment B into points with a distance of 1 pixel.
   For each point, draw a perpendicular line segment C (bounded by the body mask) and measure the length from the point to the end of segment C towards the left. Select the point on the mask contour with minimum length.
   Measure the length from the point to the end of segment C towards the right for each point and select the point with minimum length.
   Compute the 2D distance between the selected points as the output measurement

Note that where the above examples refer to drawing and measuring line segments this is for illustrative purposes and to allow visualization. In practice there is typically no need to actually draw the segments but rather the line segment locations and lengths are computed.

The body mask referenced above refers to the outer contour of the body (or equivalently of the local body part segment) as defined by the segmentation model produced by the image segmentation algorithm described previously.

The above rules for computing various measurements are given purely by way of example. The specific rules may be adapted and varied as needed. For example, the relative locations of measurement points/lines along a segment may be varied. Similar measurement rules may be implemented, adapted as needed, for other body parts, such as chest, waist, hip, forearm and upper arm (bicep) measurements.

### Perspective Correction of 2D Structure Data (Skeleton/Segmentation Model) Pinhole Camera Model

The pinhole camera model describes the relationship between the location of a 3D point in a global coordinate system, its location in the camera coordinate system and its location in the 2D image produced by the camera. Implementations use a model based on the following equations:

$$( \begin{matrix}{x}_{c} \\ {y}_{c} \\ {z}_{c}\end{matrix} ) = {R}_{c} ( \begin{matrix}{x}_{w} \\ {y}_{w} \\ {z}_{w}\end{matrix} ) + {\vec{t}}_{c} , \\ ( \begin{matrix}{x}^{′} \\ {y}^{′}\end{matrix} ) = \frac{1}{{z}_{c}} ( \begin{matrix}{x}_{c} \\ {y}_{c}\end{matrix} ) , \\ {ρ}^{2} = {x}^{′2} + {y}^{′2} , \\ {x}^{″} = {x}^{′} \frac{1 + {k}_{1} {ρ}^{2} + {k}_{2} {ρ}^{4} + {k}_{3} {ρ}^{6}}{1 + {k}_{4} {ρ}^{2} + {k}_{5} {ρ}^{4} + {k}_{6} {ρ}^{6}} + 2 {p}_{1} {x}^{′} {y}^{′} + {p}_{2} ( {ρ}^{2} + 2 {x}^{′2} ) , \\ {y}^{″} = {y}^{′} \frac{1 + {k}_{1} {ρ}^{2} + {k}_{2} {ρ}^{4} + {k}_{3} {ρ}^{6}}{1 + {k}_{4} {ρ}^{2} + {k}_{5} {ρ}^{4} + {k}_{6} {ρ}^{6}} + {p}_{1} ( {ρ}^{2} + 2 {y}^{′2} ) + 2 {p}_{2} {x}^{′} {y}^{′} , \\ ( \begin{matrix}u \\ v\end{matrix} ) = ( \begin{matrix}{f}_{x} & 0 & {c}_{x} \\ 0 & {f}_{y} & {c}_{y}\end{matrix} ) ( \begin{matrix}{x}^{″} \\ {y}^{″} \\ 1\end{matrix} ) ,$$

where
x_{w}, y_{w}, z_{w} are 3D point coordinates in global/world coordinate system;
x_{c}, y_{c}, z_{c} are 3D point coordinates in camera coordinate system;
R_{c} is 3×3 rotation matrix to convert from global coordinate system to camera coordinate system;
\vec{t}_{c} is 3×1 translation vector to convert from global coordinate system to camera coordinate system;
u, v are 2D pixel coordinates corresponding to the 3D point visible by the camera;
k₁, k₂, k₃, k₄, k₅, k₆, p₁, p₂ are camera lens distortion coefficients;
fₓ, f_{y}, cₓ, c_{y} are camera matrix parameters.
   If z_{c}23 0 then the point can't be seen by the camera. The

$${d}_{c} = {z}_{c} \sqrt{{( \frac{{x}_{c}}{{z}_{c}} )}^{2} + {( \frac{{y}_{c}}{{z}_{c}} )}^{2} + 1}$$

value is also known as 3D point depth.

### Intrinsic Camera Calibration for Smartphone

Camera matrix parameters and lens distortion coefficients together are referred to as intrinsic camera parameters. An important fact about these parameters is that for a certain camera exemplar they always remain the same regardless of any external factor. It means that once these parameters have been estimated for a camera, their values can be used in all computational models involving the camera.

Furthermore, all exemplars of a certain smartphone model share intrinsic camera parameters. Thus, one can estimate internal camera parameters for all the smartphone models being used beforehand without involving end users in the procedure.

In an implementation, the Vizario Camera (https://www.vizar.io/vizariocam/) application (available both in iOS and Android markets) is used to calibrate intrinsic camera parameters. The calibration procedure involves visualizing a supported calibration pattern (e.g., chessboard) on a computer screen and taking multiple shots of it from the smartphone camera at different angles and from different distances. The application automatically recognizes the pattern and uses recognized views in order to find the intrinsic parameters of the smartphone camera. Increased variability in views can improve calibration quality. However, other 20 software or manual calibration techniques may be used. For example, the parameters for calibration may be manually selected by an expert, based on prior experience working with smartphone cameras, camera specifications etc.

### Perspective Correction Based on Device Orientation (Gravity Vector)

The skeleton and segmentation models as initially extracted and corrected using the pinhole camera model define a representation of the subject body pose from the image in a distorted coordinate space, with the distortion being due to the device's orientation (which will typically not be oriented exactly parallel to the subject body; i.e. the image plane is tilted with respect to the plane of the body). The orientation of the device is specified by the gravity vectors supplied by the user device.

To implement the correction, the system identifies three 3×1 correction vectors, for each of the possible dimension planes (x,y,z), from the device's gravity vectors.

These vectors are then combined into one 3×3 correction matrix, R_{C}

The correction matrix is then applied to the distorted coordinate plane, transforming it. Specifically, the vector representations of the skeleton and segmentation model are transformed in this way, using the correction matrix, resulting in transformed skeleton and segmentation models.

The matrix transformation results in a corrected representation of the subject pose in the image (as defined by the skeleton and segmentation models), in which the image coordinate space has been transformed into a real world coordinate space.

### System Architecture

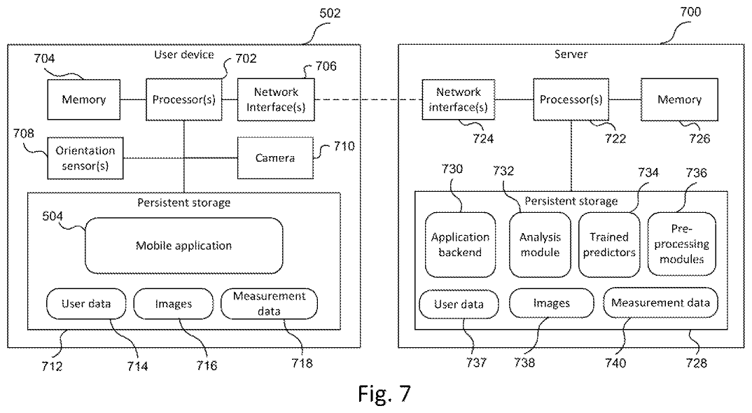

FIG. 7 illustrates a user device 502 and server 700 in accordance with an implementation. In this implementation, the functions of the application server 508 and analysis server 520 of FIG. 5 are combined in single server device 700. The user device 502, e.g. a smartphone, includes one or more processors 702 together with 20 volatile/random access memory 704 for storing temporary data and software code being executed.

A network interface 706 is provided for communication with other system components (e.g. server 700) over one or more networks (including the Internet/mobile telecommunications networks, e.g. via a mobile telephony network interface, local WiFi interface etc.).

One or more orientation sensor(s), e.g. gyroscope(s) 708 provide information on the device orientation in the form of a gravity vector. The device also includes camera system 710 which is used to acquire images of the subject.

Persistent storage 712 (e.g. in the form of FLASH memory) persistently stores required software and data, including, for example, the mobile application 504, user data 714, acquired images 716 and measurement data 718. The persistent storage also includes other software and data (not shown), such as a device operating system (e.g. Android/i0S).

The user device will include other conventional hardware and software components as known to those skilled in the art (e.g. other sensors, touch display interface etc.), and the hardware components are interconnected by memory and I/O buses.

The server 700 includes one or more processors 722 together with volatile/random access memory 726 for storing temporary data and software code being executed. A network interface 724 is provided for communication with other system components (e.g. user device 502) over one or more networks (including the Internet).

Persistent storage 728 (e.g. in the form of magnetic or FLASH based hard disk storage, optical storage media etc.) persistently stores required software and data, including, for example, an application backend 730 (implementing functions of application server 508), analysis module 732 for performing the image pre-processing and analysis (e.g. segmentation, 2D image measurement extraction etc.), and a set of trained predictors 734 for generating estimated body part measurements from 2D image measurements extracted from the image. Data maintained at the server may, for example, include user data 737, images 738 and measurement data 740. The persistent storage also includes other server software and data (not shown), such as a server operating system.

The server will include other conventional hardware and software components as known to those skilled in the art, and the hardware components are interconnected by memory and I/O buses. While a specific architecture of the user device and server is shown by way of example, any appropriate hardware/software architecture may be employed. Furthermore, functional components indicated as separate may be combined and vice versa. For example, functions of the server 700 may be divided across multiple servers, with different servers implementing different sub-functions (e.g. as shown in FIG. 5) and/or with multiple servers implementing the same functions to support greater processing capacity (e.g. to support image analysis for a large set of user devices).

It will be understood that the present innovation has been described above purely by way of example, and modification of detail can be made within the scope of the innovation.

What is claimed is:

1. A method for of determining a body measurement of a subject based on images of the subject, the method comprising:
receiving a set of images of the subject, each image depicting the subject in a respective body pose;
for each of a plurality of images of the image set, identifying for a given body part of a body of the subject an image measurement of the given body part based on the image, the image measurement comprising a distance measurement pertaining to the body part derived from the image;
inputting the image measurements determined for the plurality of images to a prediction model, the prediction model trained on training data so as to generate a predicted body measurement of the given body part based on the image measurements; and
outputting the predicted body measurement.

2. A method according to claim 1, comprising, for each image, deriving two-dimensional structure information relating to the body pose, and determining the image measurement using the structure information.

3. A method according to claim 2, wherein the structure information comprises one or both of:
a skeleton model, the skeleton model comprising one or more skeleton points and/or one or more line segments connecting skeleton points, optionally as vertices and edges of a skeleton graph; and
a segmentation model, identifying a plurality of segments of the image corresponding to respective body parts of the subject, wherein the segmentation model comprises a mesh defining segment contours of respective segments.

4. A method according to claim 3, comprising applying one or more corrections to the two-dimensional structure information in dependence on characteristics of a camera system used to obtain the image, resulting in corrected structure information, wherein the image measurement is determined using the corrected structure information.

5. A method according to claim 4, wherein the one or more corrections comprise a correction based on a camera model to correct for image distortions caused by one or more optical characteristics of the camera system.

6. A method according to claim 4, wherein the camera system is part of a user device, the one or more corrections comprising a correction based on an orientation of the user device when the image was acquired.

7. A method according to claim 6, comprising receiving device orientation information associated with the image, and applying the correction based on the device orientation information, the orientation information optionally including a gravity vector obtained using one or more sensors of the user device.

8. A method according to claim 4, comprising applying the one or more corrections to the skeleton model and the segmentation model.

9. A method according to claim 3, comprising, for each image, performing segmentation by applying a segmentation algorithm to the image to obtain the segmentation model for the image by identifying a plurality of image segments corresponding to respective body parts, and identifying the image measurement based on the segmentation model.

10. A method according to claim 9, wherein the segmentation further comprises performing a whole body segmentation to identify a whole body mask for the image, and refining the segmentation model based on the whole body mask, optionally by constraining segments in the segmentation model to an exterior body contour defined by the whole body mask.

11. A method according to claim 10, further comprising applying a hair segmentation algorithm to obtain a hair segmentation mask identifying one or more image segments corresponding to head hair, and combining the hair segmentation mask with the segmentation model and optionally the whole body mask to produce a refined segmentation model.

12. A method according claim 3, comprising identifying a segment corresponding to the given body part in the segmentation model, wherein the image measurement is determined based on the identified segment.

13. A method according to claim 12, wherein the image measurement is determined based on a two-dimensional extent of the identified segment, wherein the two-dimensional extent is optionally a width or half-width of the segment measured in relation to a longitudinal axis of the segment.

14. A method according to claim 13, wherein the image measurement is further determined based on the skeleton model, based on a part of the skeleton model corresponding to the given body part, the method optionally comprising identifying a line segment corresponding to the body part from the skeletal model, and identifying a measurement line as a line perpendicular to the line segment located at a predetermined location along the line segment and bounded by the segment contour, wherein the distance measurement is determined based on the measurement line.

15. A method according to claim 1, comprising scaling the image measurements based on a reference measurement of the body of the subject, a height of the subject, and providing the scaled image measurements as inputs to the prediction model.

16. A method according to claim 15, comprising obtaining the reference or height measurement based on user input, or based on measurement using one or more sensors of a user device, optionally using a LIDAR sensor.

17. A method according to claim 1, wherein the prediction model receives the image measurements for the given body part determined from each of the plurality of images as inputs and outputs the predicted body measurement of the body part.

18. A method according to claim 1, wherein the predicted body measurement comprises a circumference of the body part.

19. A method according to claim 1, wherein the predicted body measurement comprises a volume measurement of the body part.

20. A method according to claim 1, wherein the prediction model comprises a machine learning model trained on a set of training samples, the training samples comprising: image measurements derived from images of a plurality of subjects each in a plurality of body poses, and corresponding measured body measurements.

21. A method according to claim 1, wherein the prediction model comprises one of:
a neural network model, optionally a single-layer perceptron model or a multi-layer neural network model; and
a linear predictor model, based on a linear combination of terms, each term comprising a respective image measurement.

22. A method according to claim 1, comprising providing a plurality of trained predictor models, each trained to predict a body measurement, optionally a circumference, for a respective type of body part.

23. A method according to claim 22, comprising determining image measurements for each of a plurality of body parts based on corresponding segments in a segmentation model using images from the set of images, and obtaining and outputting a predicted body measurement for each body part using a respective one of the trained predictor models associated with that body part.

24. A method according to claim 1, comprising determining a plurality of predicted measurements of the given body part using one or more predictor models based on a plurality of image measurements obtained from the images, and deriving volume data, optionally a volume measurement, for the body part from the plurality of predicted measurements.

25. A method according to claim 24, comprising determining a plurality of circumferences of the given body part at different locations on the body part, determining an approximated three-dimensional model of the body part from the plurality of circumferences, and deriving the volume data using the approximated three-dimensional model.

26. A method according to claim 24, comprising determining derived health data from the body measurement(s) and/or volume data, optionally including a body mass index.

27. A method according to claim 1, comprising receiving the images from an application running on a user device and outputting the body measurement(s) to the application, and further comprising tracking changes in one or more body measurement(s) over multiple measurement sessions and outputting change information to a user via the application.

28. A computer readable medium comprising software code adapted, when executed by a data processing device, to perform a method according to claim 1.

29. A system having means, in a form of one or more processors with associated memory, for performing a method according to claim 1.

30. A system comprising a server system and a mobile user device having a camera system for acquiring a plurality of images of a subject and transmitting the images and optionally device orientation information to the server system, the server system configured to perform a method according to claim 1 and to output one or more body measurements to the mobile user device.
